(19) United States
(12) Patent Application Publication
(10) Pub. No.: US 2025/0011895 A1
(43) Pub. Date: Jan. 9, 2025

(54) APPARATUS AND METHOD FOR REMOVING MINERALS FROM A MINERAL CONTAINING SUBSTANCE

(71) Applicant: RIPCELL LIMITED, Peterhead (GB)

(72) Inventor: Eve Wildman, Peterhead (GB)

(21) Appl. No.: 18/713,314

(22) PCT Filed: Nov. 28, 2022
(86) PCT No.: PCT/GB2022/053007

§ 371 Date: May 24, 2024

(30) Foreign Application Priority Data
Nov. 26, 2021 (GB) 2117110.3

(51) Int. Cl. C22B 3/16; C22B 3/22; C22B 7/00; C22B 26/12

(52) U.S. Cl. CPC C22B 3/1658; C22B 3/22; C22B 7/007; C22B 26/12

(57) ABSTRACT

The present invention relates to the field of chemistry, in particular the field of acids, and methods and processes relating to increasing the concentration, and decreasing the pH, of green organic acids (and thereby increasing their leaching ability), and especially from acids obtained as the by-process of biological processes such as pot-ale and pot-ale derived acids. More particularly, the present invention relates to apparatus and methods for removing minerals from a mineral containing substance using such acids. a method for removing minerals from a mineral containing substance, the method comprising taking a primary solution having a pH below 7 and containing one or more organic acids and water; freeze separating the primary solution to remove some of the water and thereby decreasing the pH of the primary solution to form a leachant; and applying the leachant to the substance to form a leachate.

### FIELD OF THE INVENTION

The present invention relates to the field of chemistry, in particular the field of acids, and methods and processes relating to increasing the concentration, and decreasing the pH, of green organic acids (and thereby increasing their leaching ability), and especially from acids obtained as the by-process of biological processes such as pot-ale and pot-ale derived acids. More particularly, the present invention relates to apparatus and methods for removing minerals from a mineral containing substance using such acids.

### BACKGROUND TO THE INVENTION

Green, organic acids are produced from a variety of different sources, such as pot-ale derived acids from distilleries. Such acids will typically be simple acids such as acetic acid, and lactic acid.

One such process is the leaching of useful metals from end-of-life rechargeable batteries. This requires the use of concentrated acids such as hydrochloric or sulphuric acid, acids which may be difficult or expensive to obtain. There is also an environmental cost to consider, given that these specialty chemicals often have to be transported long distances to reach the site of use, and their disposal is also an environmental concern.

Electric Vehicle (“EV”) batteries manufacturing and the recycling End Of Life (“EOL”) batteries are mutually supportive industries, but at present the global EV industry lags behind that of mining fresh materials because of the relatively high cost of recycling versus mining.

### SUMMARY OF THE INVENTION

According to a first aspect of the present invention there is provided a method for removing minerals from a mineral containing substance, the method comprising:
taking a primary solution having a pH below 7 and containing one or more organic acids and water;
freeze separating the primary solution to remove some of the water and thereby decreasing the pH of the primary solution to form a leachant; and
applying the leachant to the substance to form a leachate.

The leachant may be an acid having a concentration of approximately 1M (1 mol/L−1).

The method may further comprise allowing the leachant to treat the substance for a period of time thereby forming a leachate comprising the leachant and one or more minerals taken from the substance

The period of time may be from 0 to 5 hours.

The period of time may be at least one hour.

The method may further comprise adding a base to the solution after the period of time has elapsed.

The base may be sodium hydroxide.

The pH after adding the alkali to the substance may be at least 11.

The solution and/or substance may be increased in temperature to leaching temperature. This may occur during, before or after the solution has been applied to the substance.

The leaching temperature may be in the range of 60 to 90 degrees Celsius.

The leaching temperature may be in the range of 65 to 75 degrees Celsius.

The leaching temperature may be 70 degrees Celsius.

The primary solution may be a by-product of an organic process.

The primary solution may contain acetic acid.

The primary solution may contain lactic acid.

The primary solution may contain a combination of acetic and lactic acid.

The primary solution may be pot-ale or derived from pot-ale.

The primary solution may have a starting pH of between 3 and 4.

The substance may be recycled batteries.

The batteries may be lithium-ion batteries.

The substance may be shredded batteries. This is often colloquially referred to as the “black mass”.

The substance may contain one or more of the following metals: Lithium, Nickel, Cobalt and Manganese.

The leachant may leach the one or more metals from the substance to form a leachant

The one or more metals may be leached in the form of the hydroxide form of the metal.

The method may further comprise the filtration of the leachate.

This filtration may be achieved by vacuum filtration.

The filtration may be used to filter out graphite.

The method may further comprise treating the leachate with gaseous carbon dioxide.

The gaseous carbon dioxide may precipitate lithium from the substance as lithium carbonate.

The method may further comprise monitoring the purity and concentration of metals.

This monitoring may take place on a batch basis using ICP-OES.

This monitoring may take place on a batch basis using EDX analysis.

This monitoring may take place on a batch basis using XRD analysis.

The method may further comprise the addition of a reducing agent at the leachant and substance mixing phase, or during the period of time where the substance is being subjected to the leachant.

The reducing agent may be for example hydrogen peroxide (H₂O₂) or may be in the form of naturally occurring glucose within the primary solution.

According to second aspect of the present invention there is provided apparatus adapted to carry out the method.

### BRIEF DESCRIPTION OF THE DRAWING

Embodiments of the present invention will now be described, by way of example only, with reference to the following drawings, in which:

FIG. 1 is a flowchart embodying the method of the present invention;

FIG. 2 is a graph showing concentration of acids from known sources;

FIG. 3 is a graph showing leaching efficiency against temperature for various metals; and

FIG. 4 is a graph showing leaching efficiency against time for various metals.

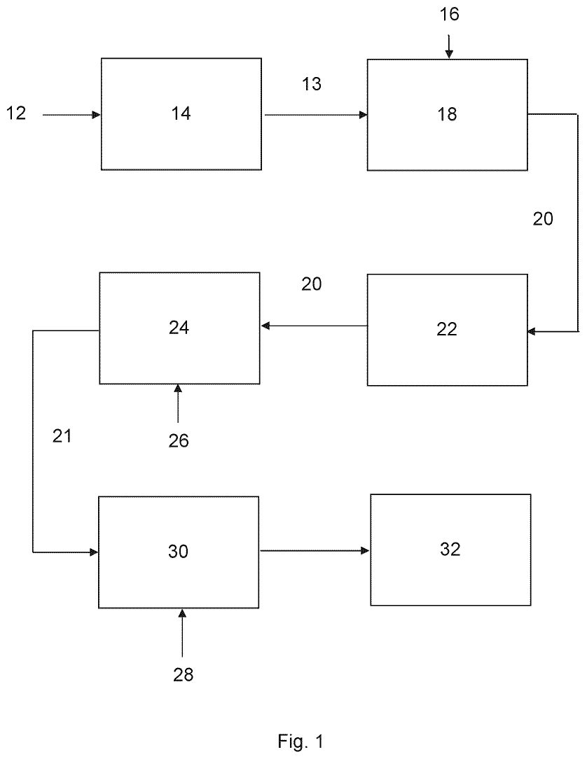

FIG. 1 depicts a method for removing minerals from a substance generally referred to as 10. The method generally comprises the steps of taking a primary solution 12 having a pH below 7 and containing one or more organic acids and water, freeze separating the solution at step 14 to remove some of the water and thereby decreasing the pH of the primary solution 12. This forms a leachant 13 having concentration of approximately 1M (1 mol/L−1) which is then applied to a substance 16 at step 18. The leachant 13 is allowed to treat the substance 16 for a period of time, from 0 to 5 hours, but in the present embodiment of least one hour.

In the present embodiment, the substance 16 is the “black mass” of ground up rechargeable batteries for recycling purposes, and the substance contains one or more of the metals Lithium, Nickel, Cobalt and Manganese.

The primary solution 12 in the present embodiment is pot-ale. Pot-ale is a by-product of spirit distillation containing lactic and/or acetic acid which has a pH of around 3 to 4.

By freeze-separating the primary solution 12, the primary solution 12 becomes a leachant 13 being an acid with a higher concentration, going from around 10 g acid per litre, (around 0.2 molar) to acid concentration of around 60 g per litre (around 1 molar).

The leachant 13 may be applied at, or heated to, around 60 to 90 degrees Celsius, more specifically around 65 to 75 degrees Celsius to a leaching temperature. The leaching temperature is specifically 70 degrees Celsius in the present embodiment.

The mixture of the leachant 13 and substance 16 is allowed to sit at the leaching temperature for a period of time, ideally between zero and five hours, but in the specific present embodiment this is a period of around one hour. In this time, the leachant 13 leaches one or more metals from the substance 16. This forms a leachate 20 i.e. a mixture of the leachant 13 and the metals present within the substance 16.

Vacuum filtration is applied to the leachate 20 to filter out graphite at step 22. Graphite is used as anodes and is present in significant quantities in the substance 16.

A base 26 is then applied at step 24 to the leachate 20. The base 26 has a pH of 11 or higher, and in the present embodiment is sodium hydroxide (NaOH) in aqueous solution form. The one or more metals will then co-precipitate in the form of the hydroxide form of the metal i.e. hydroxides (LiₓNiₓMnₓCoₓ(OH)₂). This forms a base adjusted leachate 21.

The base adjusted leachate 21 is then treated with gaseous carbon dioxide 28 at step 30. The gaseous carbon dioxide 28 causes the lithium to precipitate from the substance as lithium carbonate (Li₂CO₃). A fraction of the Lithium may remain in the solution.

The method may further comprise monitoring the purity and concentration of metals and this monitoring may take place on a batch basis using ICP-OES, EDX and XRD at step 32.

Moreover, the method may further comprise the addition of a reducing agent at the leachant 13 and substance 16 mixing phase, or during the period of time where the substance 13 is being subjected to the leachant 13. The reducing agent may be for example hydrogen peroxide (H₂O₂) or may be in the form of naturally occurring glucose within the primary solution 12.

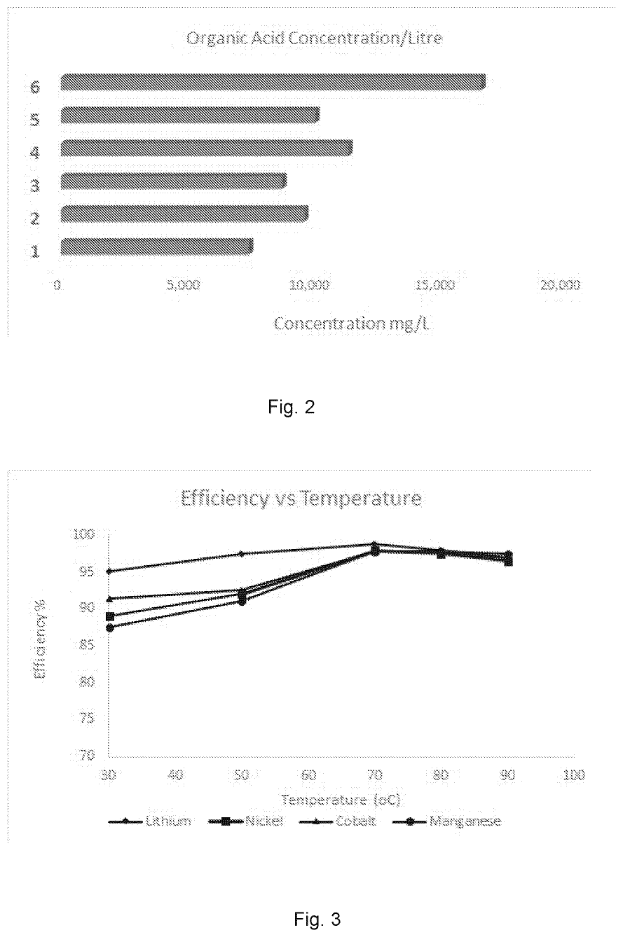

FIG. 2 shows a bar graph showing organic acid yields from six different distilleries in mg/litre, showing the general trend and variation across six such sites.

FIG. 3 shows experimental date of leaching efficiency against temperature for four different metals which are extracted in a typical process: lithium, nickel, cobalt and manganese.

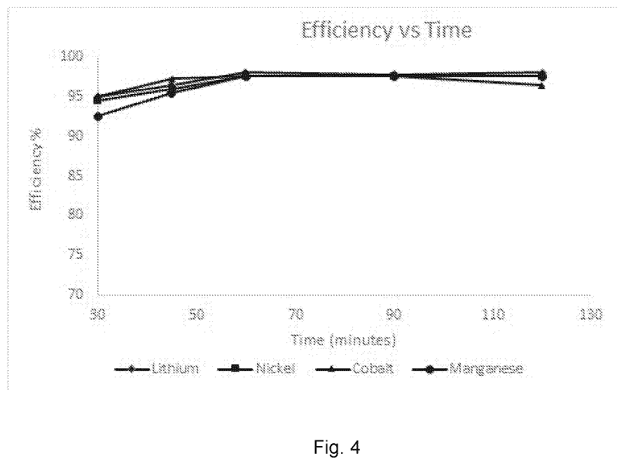

FIG. 4 shows experimental date of leaching efficiency against time for four different metals which are extracted in a typical process: lithium, nickel, cobalt and manganese.

1. A method for removing minerals from a mineral containing substance, the method comprising:
taking a primary solution having a pH below 7 and containing one or more organic acids and water;
freeze separating the primary solution to remove some of the water and thereby decreasing the pH of the primary solution to form a leachant; and
applying the leachant to the mineral containing substance to form a leachate.

2. The method of claim 1 further comprising allowing the leachant to treat the mineral containing substance for a period of time of at least one hour to form the leachate.

3. The method of claim 2 further comprising adding a base to the leachate after the period of time has elapsed.

4. The method of claim 3 wherein the base is sodium hydroxide.

5. The method of claim 1 wherein the leachant and/or mineral containing substance are increased in temperature to a leaching temperature.

6. The method of claim 5 wherein the leaching temperature is 60 to 90 degrees Celsius.

7. The method of claim 1 wherein the primary solution contains acetic and/or lactic acid.

8. The method of claim 1 wherein the primary solution is pot-ale or derived from pot-ale.

9. The method of claim 1 wherein the mineral containing substance is one or more batteries to be recycled.

10. The method of claim 9 wherein the batteries are lithium-ion batteries.

11. The method of claim 1 wherein the mineral containing substance contains one or more of the following group of minerals: Lithium, Nickel, Cobalt and Manganese.

12. The method of claim 1 further comprising filtering of the leachate.

13. The method of claim 1 further comprising treating the leachate with gaseous carbon dioxide.

14. The method of claim 1 wherein a reducing agent is added to the leachant and mineral containing substance mixture and/or to the leachate.

15. An apparatus adapted to carry out the method of claim 1.

16. The method of claim 1 further comprising adding a base to the leachate after a period of time of at least one hour has elapsed.

17. The method of claim 1 wherein the leachant and/or mineral containing substance are increased in temperature to a leaching temperature of 60 to 90 degrees Celsius and the primary solution contains acetic and/or lactic acid.

18. The method of claim 17 wherein the primary solution is pot-ale or derived from pot-ale.

19. The method of claim 18 wherein the mineral containing substance is one or more lithium-ion batteries to be recycled.
